(19) United States
(12) Patent Application Publication
(10) Pub. No.: US 2017/0027936 A1
(43) Pub. Date: Feb. 2, 2017

(54) ABL1 INHIBITOR FOR TREATING AND PREVENTING OCULAR NEOVASCULARISATION

(71) Applicant: UCL BUSINESS PLC, London (GB)

(72) Inventors: Christiana RUHRBERG, London (GB); Claudio RAIMONDI, London (GB); Alessandro FANTIN, London (GB)

(21) Appl. No.: 15/113,242

(22) PCT Filed: Jan. 20, 2015
(86) PCT No.: PCT/GB2015/050114

§ 371 Date: Jul. 21, 2016

(30) Foreign Application Priority Data
Jan. 21, 2014 (GB) 1401005.2

(51) Int. Cl. A61K 31/506; A61K 45/06; A61K 31/505

(52) U.S. Cl. CPC A61K 31/506; A61K 31/505; A61K 45/06

(57) ABSTRACT

The present invention relates to an Abelson murine leukaemia viral oncogene homolog 1 (ABL1) inhibitor for use in the treatment of ocular neovascularisation associated with a non-cancerous condition. The invention also relates to the use of an ABL1 inhibitor as a complementary therapy with VEGF or VEGF receptor inhibitor treatment and provides pharmaceutical compos itions and kits comprising one or both inhibitors.

### FIELD OF THE INVENTION

The present invention relates to compounds for use in the treatment of ocular neovascularisation. The compounds are useful in the treatment of conditions such as age-related macular degeneration, diabetic retinopathy and diabetic macular oedema.

### BACKGROUND TO THE INVENTION

Age-related macular degeneration (AMD) is a leading cause of blindness in people over 50 years of age. There are two types of macular degeneration: dry and wet AMD. In the wet form of macular degeneration, abnormal blood vessels grow in the back of the eye. Sometimes these vessels leak blood or fluid, which causes blurred or distorted vision. Without treatment, vision loss may be quick and severe.

The anti-VEGF treatments Lucentis™ and Macugen™ are approved medicines to reduce neovascularisation and oedema in wet AMD. Some have also been used for macular oedema in diabetic retinopathy and after retinal vein occlusion. Another anti-VEGF drug called Avastin™ is also widely used off-label as a more cost-effective alternative medicine to Lucentis.

These drugs are given by a monthly injection into the eye. In the case of ‘wet’ AMD, the treatments have stabilised the sight in over 90% of cases, but resulted in significant improvement in only 30% of people. For people with macular oedema following retinal vein occlusion, improvement was seen in around 60% of people and for people with diabetic retinopathy improvements were seen in only 30% of people. However, in the AMD patients, repeated eye injections can sometimes have serious side effects, including retinal detachment and cataracts. Moreover, long-term treatment with anti-VEGF has recently been suggested to also have serious effects on retinal health.

Anti-VEGF treatment also increases the risk of serious systemic side effects, such as arteriothrombotic events and even death. Finally, recent evidence suggests that ant-VEGF does not cure AMD, because oedema returns as soon as the treatment is discontinued.

There is thus a need for alternative therapies for conditions such as wet AMD and diabetic retinopathy that are not associated with the disadvantages and problems mentioned above.

### DESCRIPTION OF THE FIGURES

FIG. 1 NRP1 is dispensable for cell-matrix adhesion, but promotes FN-induced cell spreading, filopodia extension and motility of primary EC.

(A-C) Adhesion of EC to FN is not impaired by NRP1 downregulation. Immunoblotting (A), brightfield images (B) and time course of cell adhesion (C) in HDMEC transfected with control or NRP1 siRNA and plated for the indicated times on plastic dishes coated with 10 μg/ml FN. Scale bar: 150 μm.

(D-G) Actin remodelling and cell spreading of EC are impaired by NRP1, but not VEGFR2 downregulation. HDMEC transfected with control, VEGFR2 or NRP1 siRNA were plated on FN for the indicated times before immunoblotting (D) or fluorescent labelling with the F-actin marker phalloidin (green) and the nuclear counterstain DAPI (blue) (E); scale bar 20 μm. Images shown in (E′) are higher magnifications of areas indicated with dotted squares in (E). Quantification of cell area (G) of and phalloidin-stained microspikes (F) at the indicated time points; mean±SEM.

(H-J) EC motility is impaired by NRP1 downregulation. HDMEC transfected with control or NRP1 siRNA were plated on FN and observed for 200 minutes; (HI) show representative tracks, with the point of origin of each cell plotted as the point of origin;

(J) average track length; mean±SEM of 23 cells from 2 independent experiments.

(K-M) EC migration on FN is impaired by NRP1, but not VEGFR2 downregulation. HDMEC or HUVEC transfected with control, VEGFR2 or NRP1 siRNA, and Nrp1^{fl/fl} MLEC transfected with adenovirus carrying Gfp control or Cre transgenes (L) were plated on FN-coated transwells; the number of transmigrated HDMEC or HUVEC (K) or MLEC (M) was determined after 240 min; mean±SEM from >8 independent experiments for HDMEC; mean±SEM from 3 independent experiments for MLEC. Asterisks indicate statistical significance: *P<0.05; **P <0.01; ***P<0.001.

FIG. 2 NRP1 transduces ECM signals independently of VEGF165 and VEGFR2.

(A-C) Phosphoproteomic screening reveals NRP1-dependent signal transducers in EC. HDMEC were transfected with control or NRP1 siRNA before immunoblotting (A) and stimulation with VEGF165 for 10 min (B) or FN for 30 min (C) followed by phosphokinase-antibody array screening. Screening data are expressed as mean fold change in phosphorylation of the indicated proteins in siNRP1-transfected relative to control siRNA-transfected cells; error bars represent the SEM of 2 independent experiments, performed in duplicate each.

(D,E) Validation of NRP1-regulated phospho-proteins identified in the phosphoproteomic screen. HDMEC were transiently transfected with control, NRP1 or VEGFR2 siRNA and stimulated with VEGF165 (D) or plated on FN (E) for the indicated times; lysates were immunoblotted for the indicated proteins, with GAPDH serving as a loading control. Immunoblots are representative of 3 independent experiments.

FIG. 3 NRP1 promotes FN-induced PXN Y118 phosphorylation.

(A-C) Reduced pPXN levels in EC downregulated for NRP1, but not VEGFR2. HDMEC transiently transfected with control, NRP1 or VEGFR2 siRNA were plated on FN for the indicated times and immunofluorescently labelled for pPXN Y118 (green) together with phalloidin (red) and DAPI (blue) (A; scale bar 20 □m) or immunoblotted for the indicated proteins (B). The single channel for pPXN staining is shown on the right hand side of (A). (C) pPXN Y118 levels in immunoblots were quantified as pixel intensity relative to GAPDH and values expressed as mean fold change in cells transfected with si-VEGFR2 or si-NRP1 relative to cells transfected with control siRNA; mean±SEM of four independent experiments; *P<0.05.

(D) NRP1 forms a complex with pPXN in FN-stimulated EC. HDMEC were detached in serum-free medium (non-adherent, NA) or plated on FN for the indicated times and then immunoprecipitated with control IgG or NRP1 antibodies, followed by immunoblotting for NRP1 and pPXN Y118.

(E,F) NRP1 partially colocalises with pPXN at focal adhesion sites. Single confocal scans of HDMEC plated on FN for 45 min and immunofluorescently labelled with antibodies for NRP1 and pPXN Y118 and counterstained with DAPI; scale bar: 10 μm.

(F) shows a higher magnification of the area indicated with a dotted square in (E). Single channels for NRP1 and pPXN are shown adjacent to the triple stain in (E,E″) and (F′,F″), respectively. Arrowheads indicate examples of partial co-localisation.

FIG. 4 ABL1 knockdown impairs PXN Y118 phosphorylation and EC migration. (A-E) ABL1 knockdown reduces pPXN Y118 levels in EC. (A) qPCR shows that siABL1 effectively reduces Abli expression in HDMEC; Abl1 values were normalised to Actb and expressed as fold reduction in knockdown relative to control HDMEC; mean±SEM of 3 independent experiments. (B-E) Immunofluorescence labelling (BC) and immunoblotting (D,E) of HDMEC transfected with si-ABL1 or control siRNA and then plated on FN for the indicated times. In (B), pPXN Y118 (green) is shown together with phalloidin (red) and DAPI (blue) on the left hand side, and as single channel in greyscale on the right hand side; scale bar 20 μm. The pixel intensity of the pPXN signal was quantified in (C) as fold change in knockdown cells at the indicated time points relative to control cells at 0 min; mean±SEM of four independent experiments. (D,E) Immunoblotting shows that ABL1 downregulation reduces PXN, but not ERK1/2 phosphorylation; pCRKL served as readout for ABL1 downregulation and GAPDH as a loading control. The quantitation of pPXN Y118 levels as pixel intensity after densitometry is shown in (E); values are expressed as fold change in knockdown cells at the indicated time points relative to non-adherent (NA) control cells at 0 min; mean±SEM of three independent experiments.

(F) EC migration on FN is impaired by ABL1 downregulation. HDMEC transfected with control or ABL1 siRNA were plated on FN-coated transwells and the percentage of transmigrated HDMEC determined after 240 min in knockdown relative to control cells; mean±SEM in 4 independent experiments.

(G-I) ABL1 and NRP1 form a constitutive protein complex in EC and associate with pPXN in FN stimulated cells. (G,H) HEK cells were transfected with expression vectors for NRP1 and ABL1 (+) before immunoprecipitation with control IgG or ABL1 antibody and immunoblotting for NRP1 (G), or immunoprecipitation with NRP1 and immunoblotting for ABL1 (H). Non-transfected cells (−) were used as internal negative control. (I) To examine complex formation of endogenous proteins, HDMEC were detached and lysed (non-adherent, NA) or lysed after plating on FN for the indicated times; lysates were immunoprecipitated with control IgG or ABL1 antibody followed by immunoblotting for NRP1 and pPXN Y118.

(J,K) ABL1 recruits pPXN in a NRP1-dependent manner after FN stimulation. Neonatal Nrp1^{fl/fl} mice were induced with vehicle (control) or tamoxifen to delete NRP1 (J). Cells were cultured on FN before being detached and lysed (A) or lysed after plating on FN for the indicated times. Lysates were immunoprecipitated with IgG or ABL1 antibody and immunoblotted for NRP1 and pPXN Y118 (K). Asterisks indicate statistical significance: *P<0.05; **P<0.01.

FIG. 5 ABL1 kinase activity is essential for PXN Y118 phosphorylation in HDMEC in vitro and during retinal angiogenesis in vivo.

(A-C) ABL1 kinase activity is essential for PXN phosphorylation. Immunofluorescence labelling (A,B) and immunoblotting (C) of HDMEC treated with vehicle of Imatinib 30 min before and during plating on FN for the indicated times. In (A), pPXN Y118 (green) is shown together with phalloidin (red) and DAPI (blue) on the left hand side and as single channel in grey scale on the right hand side; scale bar 20 μm. pPXN pixel intensity was quantified in (B) and expressed as fold change in knockdown cells at the indicated time points relative to control cells at 0 min; mean±SEM of four independent experiments. (C) Immunoblotting confirmed that Imatinib treatment reduced pPXN Y118 levels; total ERK1/2 levels were used as a loading control.

(D) Endogenous NRP1 and ABL1 form a complex in EC. Co-immunoprecipitation of endogenous proteins from lysates of HDMEC, treated with vehicle or 10 μM Imatinib for 30 minutes, detached and plated on FN for the indicated times in the presence of Imatinib. Immunoprecipitation using ABL1 antibody was followed by immunoblotting performed for NRP1 and ABL1.

FIG. 6 NRP1 and ABL1 promote PXN phosphorylation during retinal angiogenesis in the mouse.

(A) A schematic representation of retinal angiogenesis illustrates how the vessel network (red) expands from the retinal centre towards the periphery (indicated by arrows); vessels are guided by an astrocyte-derived FN network (green) ahead of the vascular front, but the FN network is subsequently remodelled in line with the vessel pattern (red with green outline).

(B) Retinal angiogenesis in the P6 retina, illustrated by immunolabelling for FN together with the vascular marker IB4.

(C) Reduced NRP1 levels in Nrp1^{+/-} compared to Nrp1^{+/+} littermates, shown by immunoblot quantification of NRP1 protein levels in P6 lungs; VE-cadherin was used as loading control; protein levels in mutants were normalised to VE-cadherin expression and expressed as % decrease relative to wild type; mean±SEM; n≧3.

(D,E) Single confocal slices through the vascular front in retinas of Nrp1^{+/+} or Np1^{+/-} littermates mice (D) or treated with vehicle or 100 mg/kg/day Imatinib on days P4 and P5 (F). Retinas were immunostained for pPXN Y118 and 164; scale bar 15 □m. In (D′, F′), Imaris software was used to mask areas not labelled for IB4 to reveal pPXN staining in EC. (E-G) Quantification of vascular pPXN in an optical z-stack after applying a mask to isolate IB4-positive areas; mean pixel values of pPXN relative to IB4 are expressed as % of control±SEM; n=3. Asterisks indicate statistical significance: *P<0.05.

FIG. 7 ABL1 is essential for vessel spouting and branching in the retina. (A,B) Reduced vascular extension after Imatinib treatment. P6 retinal vasculature of mice treated with vehicle or Imatinib from P2 to P5 was immunolabelled for IB4; scale bar 1 mm (A). Vascular extension from the retinal centre to the vascular front is indicated with red arrows. (B) Vascular extension after Imatinib treatment was quantified as the distance of the IB4-positive front form the retinal centre relative to the retinal radius; vehicle, n=7; P2-5, n=4. Asterisks indicate statistical significance: **P<0.01.

(C,D) Reduced astrocyte, but not vascular FN deposition in Imatinib-treated retinas. P6 retinal vasculature of mice treated with vehicle or Imatinib from P2 to P5 was immunolabelled for IB4 and FN; scale bar 200 μm. Note reduced FN staining of astrocyte processes ahead of the vascular front (A), whilst the vasculature was prominently stained for FN. (D) Quantification of FN pixel intensity in Imatinib-treated retinas in 0.06 mm² areas of astrocyte networks ahead of the vascular front showed significantly reduced FN deposition (*P<0.05; fold change compared to controls; n=3 each). In contrast, FN pixel intensity in vascular areas, isolated with an IB4-guided IMARIS mask, was not significantly (ns) changed (P>0.05; FN relative to IB4 pixel intensity; n=3 each).

(E) Abnormal filopodia and sprout morphology in Imatinib-treated retinas. Higher magnification of the areas indicated with dotted squares in (C); scale bar 50 μm. The

IB4 single channel is shown in grey scale below each panel after contrast enhancement to highlight filopodia. The arrow indicates an abnormally long and wide sprout without lateral protrusions or connections; examples of abnormally thin, wavy and mis-oriented filopodia are indicated with arrowheads. Note that the interaction of tip cells with microglia (wavy arrow) is not prevented by Imatinib treatment.

(F,G) Dose-dependent decrease in vessel sprouting and branching after Imatinib treatment. (F) IB4-labelled P6 retinal vasculature of mice treated with vehicle or Imatinib by daily injections on P4 and P5 or from P2 to P5; scale bar 200 μm. Examples of abnormally long and wide sprouts without lateral protrusions or connections are indicated with arrows. (G) Quantification of filopodial bursts per vascular front length as an indicator of tip cell number, and quantification of branchpoints behind the vascular front; vehicle, n=7; P4/5, n=3; P2-5, n=4; ***P<0.001 versus control; ^{♯}P<0.05 for P4/5 versus P2-5treatment.

(E) Schematic representation of the NRP1-ABL1-PXN pathway and its synergism with known VEGF-A signalling pathways transduced by VEGFR2.

FIG. 8 Imatinib or endothelial loss of NRP1 similarly inhibit pathological angiogenesis in a mouse model of retinopathy.

(A) FN expression in the OIR model of vascular pathology. Immunostaining of a P17 wild type mouse retina for FN together with IB4 after sequential exposure to 7 days normoxia, 5 days of hyperoxia and 5 days of normoxia; the area indicated with a square is shown in higher magnification adjacent to the panel as double label and single channels.

(B) Pathological angiogenesis in the OIR model is reduced by Imatinib treatment. Immunostaining for collagen IV together with IB4 of P17 retinas from mice treated with vehicle (control) or Imatinib by daily injections after return from hyperoxia to normoxia (P13-16). Pseudocolouring highlights areas with vascobliteration (VO, white) and neovascularisation (NV, red).

(C-F) Angiogenesis in the OIR model is similarly reduced in Imatinib-treated retinas and retinas lacking NRP1 in EC. Quantitation of VO and NV area in Imatinib-treated relative to vehicle-injected littermates (C,D; n=8) and in tamoxifen injected Nrp1^{fl/fl} mice lacking or expressing Pdgfb-iCre-ERT2-Egfp (E,F; fold change; n=4); asterisks indicate statistical significance: *P<0.05; **P<0.01). Scale bar (A,B) 1 mm.

### SUMMARY OF ASPECTS OF THE INVENTION

The present inventors have identified a novel pro-angiogenic pathway involving neuropilin 1 (NRP1) and Abelson murine leukaemia viral oncogene homolog 1 (ABL1) and have determined that ocular angiogenesis is inhibited by treatment with an ABL1; inhibitor.

Thus in a first aspect the present invention provides an Abelson murine leukaemia viral oncogene homolog 1 (ABL1) inhibitor for use in the treatment and/or prevention of ocular neovascularisation associated with a non-cancerous condition.

The non-cancerous condition may be selected from the following list: age-related macular degeneration, diabetic macular oedema, diabetic retinopathy, retinal vein occlusion, retinopathy of prematurity, ocular histoplasmosis, macular telangiectasia, choroidal neovascularisation and pathologic myopia macular oedema.

In particular, the ABL1 inhibitor may be for use in the treatment and/or prevention of age-related macular degeneration, diabetic retinopathy or diabetic macular oedema.

The inhibitor may inhibit angiogenesis.

The inhibitor may inhibit endothelial cell actin remodelling and/or endothelial cell migration.

The inhibitor may inhibit Neuropilin 1 (NRP1)-dependent signalling.

The inhibitor may inhibit paxillin (PXN) phosphorylation.

The inhibitor may be selected from the following group: Imatinib, nilotinib, dasatinib, bosutinib, ponatinib, bafetinib and GNF-5. The inhibitor may be, for example, Imatinib or GNF-5.

The inhibitor may be used in combination with a vascular endothelial growth factor (VEGF) or VEGF receptor (VEGFR) inhibitor.

The VEGF or VEGF receptor inhibitor may inhibit the binding of VEGF to VEGF receptor or signalling of VEGF through VEGF receptor.

Examples of VEGF inhibitors include Avastin™, Lucentis™, Macugen™ or Eylea™

Examples of VEGF receptor inhibitors include Levatinib™, Motesanib™, Pazopanib™, Regorafenib™.

The ABL inhibitor and the VEGF/VEGFR inhibitor may be administered simultaneously, separately or sequentially.

In a second aspect, the present invention provides a pharmaceutical composition comprising an ABL1 inhibitor for use in the treatment and/or prevention of ocular neovascularisation associated with a non-cancerous condition.

The composition may also comprise a pharmaceutically acceptable excipient and/or carrier.

The composition may also comprise a VEGF/VEGFR inhibitor.

In a third aspect, the present invention provides a kit comprising an ABL1 inhibitor and a VEGF/VEGFR inhibitor for use in the treatment and/or prevention of ocular neovascularisation associated with a non-cancerous condition.

The VEGF/VEGFR inhibitor and the ABL1 inhibitor may be for separate, subsequent or simultaneous administration.

In a fourth aspect, the present invention provides a method for the treatment and/or prevention of ocular neovascularisation associated with a non-cancerous condition in a subject which comprises the step of administering an ABL1 inhibitor to the subject.

In a fifth aspect, the present invention provides the use of an ABL1 inhibitor in the manufacture of a medicament for treating and/or preventing ocular neovascularisation associated with a non-cancerous condition in a subject.

### DETAILED DESCRIPTION

ABL1

Abelson murine leukaemia viral oncogene homolog 1 (ABL1) is a protein that, in humans, is encoded by the ABL1 gene.

ABL-family proteins comprise a highly conserved branch of the tyrosine kinases. Each ABL protein contains an SH3-SH2-TK (Src homology 3Sic homology 2 tyrosine kinase) domain cassette, which confers autoregulated kinase activity and is common among nonreceptor tyrosine kinases. This cassette is coupled to an actin-binding and -bundling domain, which makes ABL proteins capable of connecting phosphoregulation with actin-filament reorganization. Vertebrates have evolved two ABL paralogs, ABL1 and ABL2. Several types of posttranslational modifications control ABL catalytic activity, subcellular localization, and stability, with consequences for both cytoplasmic and nuclear ABL functions. Binding partners provide additional regulation of ABL catalytic activity, substrate specificity and downstream signalling.

ABL1 is involved in a large number of cell signalling pathways. It is subject to autophosphorylation and to phosphorylation by members of the SRC family of tyrosine kinases and by platelet-derived growth factor receptor (PDGFR). The activity of ABL1 is further regulated by a range of other post-translational modifications.

A number of downstream targets for ABL1 have been reported and ABL1 signalling is involved in a variety of biochemical and cellular functions including actin binding, bundling and remodelling, cell motility and adhesion, receptor endocytosis and autophagy and DNA damage response and apoptosis.

Both vertebrate Abl1 and Abl2 are required for normal development. 75% of Abl1^{-/-} mouse mutants die within 2 weeks after birth and the remaining 25% reaching adulthood are susceptible to infections. Abl2^{-/-} mouse mutants are runts at three weeks after birth, but by 6 weeks are of similar size as wild type littermate. Intercrosses of Abl1 and Abl2 mutants reveal overlapping roles for Abl1 and Abl2 during development. Abl1+/− Abl2+/− heterozygous mice are born and survive normally, whilst 60% of Abl1+/− Abl2−/− mice survive to adulthood and all Abl1−/− Abl2+/− die embryonically at 15.5 days post coitum. Abl1+/− Abl2+/− heterozygous mice with disruptions in either of the Abl genes display a number of development defects including defective haematopoiesis and low viability, osteoporosis, decreased systolic blood pressure, cardiac hyperplasia and impaired development and responsiveness of B cells and T cells.

Mutations in the ABL1 gene are associated with chronic myelogenous leukaemia (CML). In CML, the gene is activated by being translocated within the BCR (breakpoint cluster region) gene on chromosome 22. This new fusion gene, BCR-ABL, encodes an unregulated, permanently active cytoplasm-targeted tyrosine kinase that activates mediators of the cell cycle regulation system, leading to a clonal myeloproliferative disorder. This translocation has also been observed in other cancers and leukaemias.

The present inventors have determined that ABL1 functions downstream of neuropilin 1 (NRP1) in a pro-angiogenic pathway in endothelial cells (see FIG. 7H). Activation of ABL1 via NRP1 causes ABL1 to activate the focal adhesion related protein paxillin (PXN) and promote endothelial cell motility and angiogenesis.

Neuropilin 1 (NRP1)

NRP1 is a non-catalytic receptor for the VEGF165 isoform of VEGF-A that complexes with VEGFR2 to potentiate signal transduction. Specifically, the NRP1 cytoplasmic tail recruits a trafficking complex that directs VEGFR2 along an endocytic pathway that prevents receptor dephosphorylation to augment mitogen-activated protein kinase (MAPK) signalling via ERK1 and ERK2. This NRP1 function is essential for arteriogenesis, which depends on luminal vessel growth, but dispensable for angiogenesis, which is driven by vessel sprouting, branching and fusion.

The present inventors have determined that NRP1-dependent extra-cellular matrix (ECM) signalling stimulates phosphorylation of the integrin target PXN in endothelial cells. This was determined to occur through ABL1 and independently of VEGFR2. This signalling was associated with cellular migration of endothelial cells and vascular sprout extension and branching during both physiological and pathological angiogenesis.

Paxillin (PXN)

Paxillin is a signal transduction adaptor protein. The C-terminal region of paxillin contains four LIM domains that target paxillin to focal adhesions, possibly through a direct association with the cytoplasmic tail of beta-integrin. PXN is activated by phosphorylation. Phosphorylated PXN (pPXN) is recruited to focal adhesions to promote their turnover during cell migration and PXN has been reported to be involved in cell adhesion, motility and polarity.

The N-terminal region of paxillin is rich in proteinprotein interaction sites. The proteins that bind to paxillin are diverse and include protein tyrosine kinases, such as SRC and focal adhesion kinase (FAK), structural proteins, such as vinculin and actopaxin, and regulators of actin organization, such as COOL/PIX and PKL/GIT. Paxillin is tyrosine-phosphorylated by FAK, SRC and ABL1 upon integrin engagement or growth factor stimulation, creating binding sites for the adapter protein CRK.

ABL1 Inhibitors

An ABL1 inhibitor is capable of reducing or decreasing the expression or activity of ABL1.

The inhibitor may be specific for ABL1, or may act on both ABL1 and ABL2. Due to structural similarities of ABL1 and ABL2, inhibitors targeting ABL1 may also target ABL2. Since the kinases have overlapping functions, their simultaneous inhibition but may actually benefit anti-angiogenic therapies.

The ABL1 inhibitor for use according to the present invention is an inhibitor that is capable of preventing or reducing the activity of ABL1 such that angiogenesis and/or neovascularisation are inhibited. For example the inhibitor may inhibit endothelial cell actin remodelling and/or migration.

The ABL1 inhibitor for use according to the present invention inhibits NRP1-dependent signalling through ABL1. The inhibitor may prevent or reduce NRP1-dependent activation of ABL1 by inhibiting binding between NRP1 and ABL1 or by inhibiting the activation or activity of ABL1 (i.e. by inhibition of phosphorylation of ABL1).

The ABL1 inhibitor for use according to the present invention may inhibit ABL1-dependent phosphorylation of PXN. For example the inhibitor may be a small molecule inhibitor that competitively binds the active site of ABL1 and prevents it from phosphorylating PXN. The inhibitor may be an allosteric inhibitor that prevents ABL1 adopting an active conformation or interacting with PXN.

The ABL1 inhibitor may decrease the level of ABL1 protein in the cell such that the level of signalling through the NRP1-ABL1 pathway is reduced and the level of phosphorylated PXN is reduced.

The ABL1 inhibitor for use according to the present invention may reduce the level of ABL1 activity to 90, 75, 50, 25, 20, 15, 10, 5, 2, 1, 0.5, 0.2, 0.1, 0.01% or less compared to the activity of ABL1 in the absence of the inhibitor.

A range of small molecule BCR-ABL1 tyrosine kinase inhibitors (TKI) have been developed as therapies for CML and are currently in either clinical use or clinical trials. Such inhibitors include the first-generation Imatinib and second-generation inhibitors such as nilotinib, dasatinib, bosutinib, ponatinib and bafetinib.

Imatinib binds to the kinase domain of ABL only when the domain adopts the inactive or “closed” conformation. This is where the glycine-rich, P-binding phosphate loop (P-loop) folds over the ATP binding site and the activation-loop adopts a conformation in which it occludes the substrate binding site and disrupts the ATP phosphate binding site to block the catalytic activity of the enzyme.

Nilotinib is a phenylamino-pyrimidine derivative that is structurally related to Imatinib. It was developed based on the structure of the ABL-Imatinib complex to address the need associated with Imatinib intolerance and resistance. Small changes were made on the Imatinib molecule to make it more potent and selective as a BCR-ABL inhibitor.

Dasatinib binds to ABL with less stringent conformational requirements than Imatinib so it exhibits increased potency but reduced selectivity. Dasatinib exclusively binds the active conformation of ABL kinase, contrary to most TKIs.

Bosutinib is based on a quinoline scaffold and inhibits SRC, ABL and a wide range of both tyrosine and serine-threonine kinases.

Ponatinib is an orally active BCR-ABL TKI, which was developed to be effective against the Imatinib-resistant T315I BCR-ABL mutation.

Bafetinib was developed as a more potent drug than Imatinib, with efficacy against various point mutations in the BCR-ABL kinase. Due to the structural similarities of Imatinib and bafetinib, their binding to BCR-ABL is also quite similar. Bafetinib is used in TKI therapy, as it is effective against most Imatinib resistant mutations.

Allosteric inhibitors of ABL kinases, such as N-hydroxyethyl carboxamide analogs, have also been developed. These bind to the myristate-binding site located near the C-terminus of the kinase domain to induce a bent conformation of the al helix that facilitates stabilization of an inhibited conformation. For example N-(2-Hydroxyethyl)-3-(6-(4(trifluoromethoxy)phenylamino)pyrimidin-4-yl)benzamide (ABL Inhibitor III/GNF-5) is a cell-permeable N-hydroxyethyl carboxamide analogue that exhibits in vivo efficacy in suppressing the proliferation of BCR-ABL-expressing Ba/F3 and bone marrow cells in murine xenograft models of leukaemia.

The ABL1 inhibitor for use in the present invention may be an inhibitor described above or another small molecule inhibitor of ABL1.

For example the ABL1 inhibitor may be Imatinib or GNF-5.

Imatinib has previously been reported to target PDGFR-α, which has a role in the viability and proliferation of smooth muscle cells. It is important to note that, in the present invention, inhibitors such as Imatinib are used to target the NRP1-ABL1 pathway, not the PDGFR-α pathway; and to target endothelial cells not smooth muscle cells. It is perhaps surprising that Imatinib does target endothelial cells, because previous reports have suggested that it does not have such an effect (Rocha et al. 2007; Angiogenesis 10:279-286).

The ABL1 inhibitor for use in the present invention may be a nucleic acid based inhibitor that reduces ABL1 kinase activity and/or ABL1 expression level. For example it may be an anti-sense nucleic acid, a siRNA or a miRNA.

Methods for determining the activity of a potential ABL1 inhibitor are well known in the art. For example, western blotting may be used to determine the level of phosphorylated ABL1 targets in the presence of the inhibitor compared to its absence. The level of pPXN may therefore be determined in the presence and absence of a potential ABL1 inhibitor. It is also possible to determine the level of phosphorylation of ABL1 targets by methods known in the art, such as colorimetric, radioactive or fluorometric detection of the phosphorylated target. Phospho-specific antibodies can also be used with methods such as ELISA, Western blot and flow cytometry.

Diseases Associated With Ocular Neovascularisation

The present invention relates to an ABL1 inhibitor for use in the treatment and/or prevention of ocular neovascularisation.

Neovascularisation is the abnormal proliferation of blood vessels in a tissue. In the case of ocular neovascularisation, this is the abnormal proliferation of blood vessels in the eye.

Retinal and choroidal vascular diseases constitute the most common causes of moderate and severe vision loss in developed countries. They can be divided into retinal vascular diseases, in which there is leakage and/or neovascularization (NV) from retinal vessels, and subretinal NV, in which new vessels grow into the normally avascular outer retina and subretinal space. The first category of diseases includes diabetic retinopathy, retinal vein occlusions, retinopathy of prematurity and neovascular glaucoma, and the second category includes neovascular age-related macular degeneration (AMD), ocular histoplasmosis, pathologic myopia and other related diseases.

The ocular neovascularisation may be associated with an eye disease, such as age-related macular degeneration, diabetic retinopathy, diabetic macular oedema, retinal vein occlusion, retinopathy of prematurity, ocular histoplasmosis or pathologic myopia macular oedema.

Hence, the present invention provides an ABL1 inhibitor for use in the treatment and/or prevention of one or more of the eye diseases mentioned above.

The ocular neovascularisation treated as described herein may be associated with a non-cancerous condition, such as the conditions mentioned above.

The ocular neovascularisation treated by the inhibitor described herein is NOT associated with a cancerous condition. It is not associated with the uncontrolled growth of malignant cells, nor is it attributable to tumour related angiogenesis. The condition is not a cancerous condition such as leukaemia. The use of the present invention is to target non-cancerous ocular blood vessel growth, not tumour vessel growth.

Age-Related Macular Degeneration

Age-related macular degeneration (AMD) is the leading cause of severe visual loss in persons over 65 years old. In contrast to ROP and PDR, in which neovascularisation emanates from the retinal vasculature and extends into the vitreous cavity, AMD is associated with neovascularisation originating from the choroidal vasculature and extending into the subretinal space. Choroidal neovascularisation causes severe visual loss in AMD patients because it occurs in the macula, the area of retina responsible for central vision. The stimuli that lead to choroidal neovascularisation are not understood. Laser ablation of the choroidal neovascularisation may stabilize vision in selected patients. However, only 10% to 15% of patients with neovascular AMD have lesions judged to be appropriate for laser photocoagulation according to current criteria.

AMD results in a loss of vision in the centre of the visual field (the macula) because of damage to the retina. It occurs in “dry” and “wet” forms and is a major cause of blindness and visual impairment in older adults (>50 years).

The clinical progression of AMD is characterised in stages according to changes in the macula. The hallmark of early AMD is drusen, which are accumulations of extracellular debris underneath the retina and appear as yellow spots in the retina on clinical exam and on fundus photographs. Depending on the size and quantity of the drusen and the simultaneous presence of other AMD features such as pigmentation changes of the retina, patients with early AMD may progress to advanced AMD. When patients are diagnosed with advanced AMD, which has two types that include geographic atrophy AMD and neovascular AMD, they typically have had noticeable vision loss.

Drusens are categorised by sizes as small (<63 μm), medium (63-124 μm) and large (>124 μm). They are also considered as hard or soft depending on the appearance of their margins on ophthalmological examination. While hard drusens have clearly defined margins, soft ones have less defined and fluid margins. The Age-related Eye Disease Study (AREDS) fundus photographic severity scale is one of the main classification systems used for this condition.

“Dry” or nonexudative AMD is characterised by discrete regional loss of the retinal pigment epithelium. Wet AMD results from the abnormal growth of blood vessels from the choriocapillaris (choroidal neovascularisation) through Bruch's membrane. The fragility of the blood vessels and inflammatory processes lead to subretinal haemorrhages and fibrovascular scarring. This process can occur de novo or as a progression of dry AMD. Although wet AMD is only responsible for 15% of the total AMD, it is responsible for more than 80% of AMD-related severe visual loss and blindness.

Diabetic Retinopathy and Macular Oedema

Ocular manifestations of diabetes affect up to 80 percent of all patients who have had diabetes for 10 years or more, and 10 percent of diabetic patients will suffer from vision loss related to macular oedema.

Diabetic macular oedema (DME) is caused by leaking macular capillaries and is the most common cause of visual loss in both proliferative and non-proliferative diabetic retinopathy.

In the first stage of diabetic retinopathy, which is called non-proliferative diabetic retinopathy (NPDR), macular oedema may occur in which blood vessels leak contents into the macular region. The symptoms of macular oedema are blurring, darkening or distortion of vision.

In the second stage of diabetic retinopathy, abnormal neovascularisation occurs at the back of the eye as a part of proliferative diabetic retinopathy (PDR). These new vessels are fragile due to their formation in a hypoxic environment and can easily burst and bleed, leading to blurred vision and possible retinal damage. Fibrovascular proliferation can cause tractional retinal detachment and the new blood vessels can grow into the angle of the anterior chamber of the eye and cause neovascular glaucoma.

Diabetic retinopathy is the leading cause of blindness in adults of working age. In persons with diabetes mellitus, retinal capillary occlusions develop, creating areas of ischemic retina. Retinal ischemia serves as a stimulus for neovascular proliferations that originate from pre-existing retinal venules at the optic disk or elsewhere in the retina posterior to the equator. Severe visual loss in proliferative diabetic retinopathy (PDR) results from vitreous haemorrhage and tractional retinal detachment. Again, laser treatment (panretinal photocoagulation to ischemic retina) may arrest the progression of neovascular proliferations in this disease but only if delivered in a timely and sufficiently intense manner. Some diabetic patients, either from lack of ophthalmic care or despite adequate laser treatment, go on to sustain severe visual loss secondary to PDR. Vitrectomy surgery can reduce, but not eliminate, severe visual loss in this disease.

Retinopathy of Prematurity (ROP)

Retinopathy of prematurity (ROP) occurs in premature neonates who have received intensive care in incubators with a hyperoxic atmosphere. Normally, retinal vascularisation occurs in utero, so that, at term, the medial portion of the retina is fully vascularised, but the lateral portion is only incompletely vascularised. If a pre-term infant is treated with oxygen in an incubator, the oxygen may lead to constriction of retinal blood vessels and the regression of preformed blood vessels. This can cause a lack of oxygen (ischemia) in the retina after the baby is returned to normoxic room air. This leads to the production of molecules, for example VEGF, which cause the growth of new blood vessels. Abnormal new proliferating vessels can develop at the juncture of vascularised and avascular retina. These abnormal new vessels can grow from the retina into the vitreous, resulting in haemorrhage and tractional detachment of the retina. Although laser ablation of avascular peripheral retina may halt the neovascular process if delivered in a timely and sufficient manner, some premature babies nevertheless go on to develop retinal detachment. Surgical methods for treating ROP-related retinal detachments in neonates have limited success at this time because of unique problems associated with this surgery, such as the small size of the eyes and the extremely firm vitreoretinal attachments in neonates.

Post-Retinal Vein Occlusion

Retinal vein occlusions (RVOs) are the second most common type of retinal vascular disorder after diabetic retinal disease. They can occur at almost any age (although typically in middle to later years—most in those aged over 65 years) and their severity ranges from asymptomatic to a painful, blind eye. Occlusion of the retinal venous system by thrombus formation is the most common cause but other causes include disease of the vein wall and external compression of the vein.

Ocular Histoplasmosis

Ocular histoplasmosis is a syndrome affecting the eye, which is characterised by peripheral atrophic chorioretinal scars, atrophy or scarring adjacent to the optic disc and maculopathy. The loss of vision is caused by choroidal neovascularisation. The condition is thought to be caused by the fungus Histoplasma capsulatum.

Neovascular Glaucoma

Rubeosis iridis is a medical condition of the iris of the eye in which new abnormal blood vessels (i.e. neovascularisation) are found on the surface of the iris and grow into the angle of the eye. These blood vessels eventually go through fibrosis, which closes the normal physiologic anatomy of the angle. The closing of the angle prevents fluid from leaving the eye, resulting in an increase in intraocular pressure. This is called neovascular glaucoma.

Treatment and Prevention

The ABL1 inhibitor is for use according to the present invention in order to treat or prevent diseases associated with ocular neovascularisation, such as neovascular (wet) AMD, diabetic retinopathy or macular oedema.

When used for the prevention of diseases such as wet AMD, diabetic retinopathy or macular oedema, the invention relates to the prophylactic use of the ABL1 inhibitor. In this aspect the inhibitor may be administered to a subject who has not yet contracted wet AMD, diabetic retinopathy or diabetic macular oedema and/or who is not showing any symptoms of the disease to prevent or impair the cause of the disease or to reduce or prevent development of at least one symptom associated with the disease. The subject may have a predisposition for, or be thought to be at risk of developing, a disease such as wet AMD, diabetic retinopathy or macular oedema.

When used for the treatment of diseases such as wet AMD, diabetic retinopathy or diabetic macular oedema, the invention relates to the therapeutic use of the ABL1 inhibitor. Herein the inhibitor may be administered to a subject having an existing disease or condition in order to lessen, reduce or improve at least one symptom associated with the disease and/or to slow down, reduce or block the progression of the disease.

Angiogenesis and Neovascularisation

Angiogenesis, the sprouting of pre-existing vessels to form new vessels, is a process integral to ocular neovascularisation. For new blood vessel sprouts to form, mural cells (pericytes) are first removed from the branching vessel. Endothelial cell basement membrane and extracellular matrix are then degraded and remodelled by specific proteases such as matrix metalloproteinases, before new matrix is laid down. This new matrix, coupled with soluble growth factors, fosters the migration and proliferation of endothelial cells. After sufficient endothelial cell division and migration has occurred, endothelial cells form tube-like structures. Mural cells (pericytes in the microvasculature, smooth muscle cells in larger vessels) are recruited to the abluminal surface of the endothelium, and vessels not covered by mural cells regress. Blood flow is then established in the new vessel.

Under normal circumstances, angiogenesis is a highly ordered process under tight regulation, because it requires inducing quiescent endothelial cells in a monolayer to divide and spread to expand the vascular network only to the extent that is appropriate for growing tissues. Many positively and negatively acting factors influence angiogenesis; among these are soluble polypeptides, cell-cell and cell-matrix interactions and hemodynamic effects.

Vascular Endotheilial Growth Factor (VEGF)

Vascular endothelial growth factor (VEGFNEGF-A) is a well-characterised angiogenic factor and the main, dominant inducer of blood vessel growth. The broad term VEGF covers a number of protein isoforms from that result from alternate splicing of mRNA from a single, 8-exon, VEGFA gene. The different isoforms are referred to according to the number of amino acids in the mature protein (denoted VEGFₓₓₓ). For example, alternate splicing of exon 6 and 7 alters their heparin-binding affinity and amino acid number. In addition, VEGF isoforms differ by alternative splicing of their terminal exon (exon 8)—whereby isoform formed from the proximal splice site are denoted VEGFₓₓₓ and isoforms formed from the distal splice site (VEGF)ₓₓₓb) (in humans: VEGF₁₂₁, VEGF₁₂₁b, VEGF₁₄₅, VEGF₁₆₅, VEGF₁₆₅b, VEGF₁₈₉, VEGF₂₀₆). Other VEGF proteins (VEGF-B/C/D) exist; however, their role in ocular neovascularisation is less well characterised than that of VEGF-A.

In vitro, VEGF has been shown to stimulate endothelial cell mitosis and cell migration. VEGF is also a vasodilator and increases microvascular permeability. It is integral to the process of angiogenesis and functions to increase migration of endothelial cells, increase mitosis of endothelial cells, increase methane monooxygenase activity and increase αvβ3 integrin activity. As such it is critically important for the creation of the blood vessel lumen and vasodilation.

VEGF family members stimulate cellular responses by binding to tyrosine kinase receptors (the VEGFRs) on the cell surface, causing them to dimerise and become activated through transphosphorylation. The VEGF receptors have an extracellular portion consisting of 7 immunoglobulin-like domains, a single transmembrane spanning region and an intracellular portion containing a split tyrosine-kinase. VEGF-A binds to VEGFR-1 (Flt-1) and VEGFR-2 (KDR/Flk-1), however, VEGFR-2 is reported to mediate almost all of the known cellular responses to VEGF. VEGFR-2 receptor signalling is transduced through a wide range of secondary messengers and effectors including SRC, PI3K, AKT/PKB, RAC, FAK, MAPK (p38/p42/44), PLCy, RAS/REF and MEK. The function of VEGFR-1 is less well-defined, although it is thought to modulate VEGFR-2 signalling.

Anti-VEGF therapies are important in the treatment of certain cancers and in ocular neovascular diseases such as wet AMD. Anti-VEGF inhibitors may be monoclonal antibodies such as bevacizumab (Avastin®), antibody derivatives such as ranibizumab (Lucentis®), pegaptanib (Macugen®) and aflibercept (Eylea®), or orally-available small molecules that inhibit the tyrosine kinases stimulated by VEGF: lapatinib, sunitinib, sorafenib and axitinib. Anti-VEGF treatment for ocular diseases is presently provided by ocular injections with monoclonal antibody or antibody derivatives.

Antibodies against the VEGF receptor have also been developed. They may inhibit VEGFR-1, VEGFR-2 and/or VEGFR-3. Some VEGFR antagonists, for example lenvatinib, motesanib, are under investigation for treating various cancers. Pazopanib was approved for renal cell carcinoma in 2009. Regorafenib was approved for colorectal cancer in September 2012.

The present inventors have determined that ECM interactions with NRP1, and subsequent signalling via ABL1, provide a novel pro-angiogenic pathway that is independent of VEGF and VEGFR-2. As such the ABL1 inhibitor for use according to the present invention may be used in combination with a VEGF/VEGFR inhibitor.

The VEGF/VEGFR inhibitor may inhibit the binding of VEGF to a VEGF receptor (VEGFR-2 and/or VEGFR-1). For example, the VEGF inhibitor may be an antibody-based inhibitor that binds VEGF such as bevacizumab, ranibizumab or an aptamer such as pegaptanib or a fusion protein such as aflibercept.

The VEGFR inhibitor may be a small molecule inhibitor or an antibody, such as levatinib, motesanib, pazopanib or regorafenib, which binds VEGFR-1, -2 and/or -3. A VEGFR inhibitor may inhibit VEGF binding and/or signalling of VEGF through VEGF receptor.

The ABL1 and VEGF/VEGFR inhibitors may be used simultaneously, sequentially or separately. For example, they may be in the same composition or formulation and administered at the same time.

Alternatively the ABL1 and VEGF/VEGFR inhibitors may be present in separate compositions or formulations. Herein the inhibitors may be administered sequentially (i.e. administration of one inhibitor followed within 5 minutes, 1 hour, 24 hours, 48 hours, 1 week or 1 month by administration of the second). This process may be repeated for multiple doses of each inhibitor.

Separate administration of one inhibitor may be performed before treatment, when treatment has begun or after it is completed.

Pharmaceutical Composition

The present invention also provides a pharmaceutical composition comprising an ABL1 inhibitor for use in the treatment and/or prevention of neovascular age-related macular degeneration, diabetic retinopathy or diabetic macular oedema.

The ABL1 inhibitor in the pharmaceutical composition is an ABL1 inhibitor as defined above.

The ABL1 inhibitor may be administered with a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s) and other carrier agents.

The pharmaceutical composition may also comprise a VEGF/VEGFR antagonist as defined above.

Administration

The ABL1 inhibitor for use according to the present invention may be administered to a subject systemically, thus avoiding some of the disadvantages associated with the ocular injection of present therapies.

The administration of the ABL1 inhibitor can be accomplished using any of a variety of routes that make the active ingredient bioavailable. For example, the ABL1 inhibitor can be administered by oral and parenteral routes, intraperitoneally, intravenously, subcutaneously, transcutaneously or intramuscularly.

The ABL1 inhibitor may alternatively be administered locally, for example into the eye or eye region.

The ABL1 inhibitor may be delivered intraocularly, for example by intraocular injections. This may be the route of choice when used in combination with another therapy that is administered by this route, for example when used in combination with a VEGF inhibitor.

Typically, a physician will determine the actual dosage that is most suitable for an individual subject and it will vary with the age, weight and response of the particular patient. The dosage is such that it is sufficient to reduce and/or prevent angiogenesis and/or neovascularisation. The dosage is such that it is sufficient to stabilise or improve symptoms of the disease associated with ocular neovascularisation, such as wet AMD or diabetic retinopathy and macular oedema.

Kit

The present invention also provides a kit comprising an ABL1 inhibitor for use in the treatment and/or prevention of a disease associated with ocular neovascularisation, such as neovascular age-related macular degeneration, diabetic retinopathy or diabetic macular oedema.

The kit comprises an ABL1 inhibitor as defined above.

The kit may also comprise a VEGF inhibitor and/or pharmaceutical composition as defined above.

Method

The present invention further relates to a method for the treatment and/or prevention of a disease associated with ocular neovascularisation such as neovascular age-related macular degeneration or diabetic retinopathy and macular oedema which comprises the step of administering an ABL1 inhibitor to a subject.

The method comprises the use of an ABL1 inhibitor as defined above.

The method may also comprise the use of a VEGF inhibitor and/or a pharmaceutical composition as defined above.

The present invention also relates to use of an ABL1 inhibitor in the manufacture of a medicament for treating and/or preventing neovascular age-related macular degeneration, diabetic retinopathy macular telangiectasia, choroidal neovascularisation or diabetic macular oedema in a subject.

The invention will now be further described by way of Examples, which are meant to serve to assist one of ordinary skill in the art in carrying out the invention and are not intended in any way to limit the scope of the invention.

### EXAMPLES

### Example 1

### NRP1 Promotes Spreading and Actin Remodelling of FN-Stimulated EC Independently of Roles in Cell Adhesion and VEGFR2

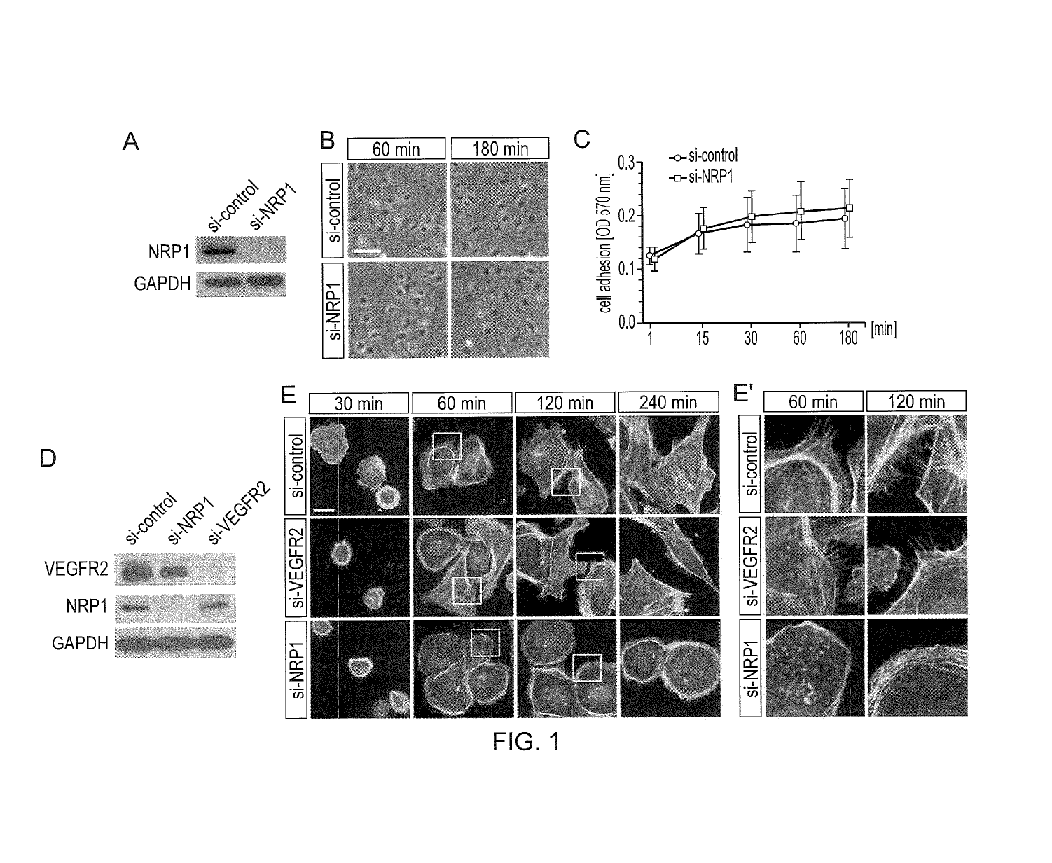

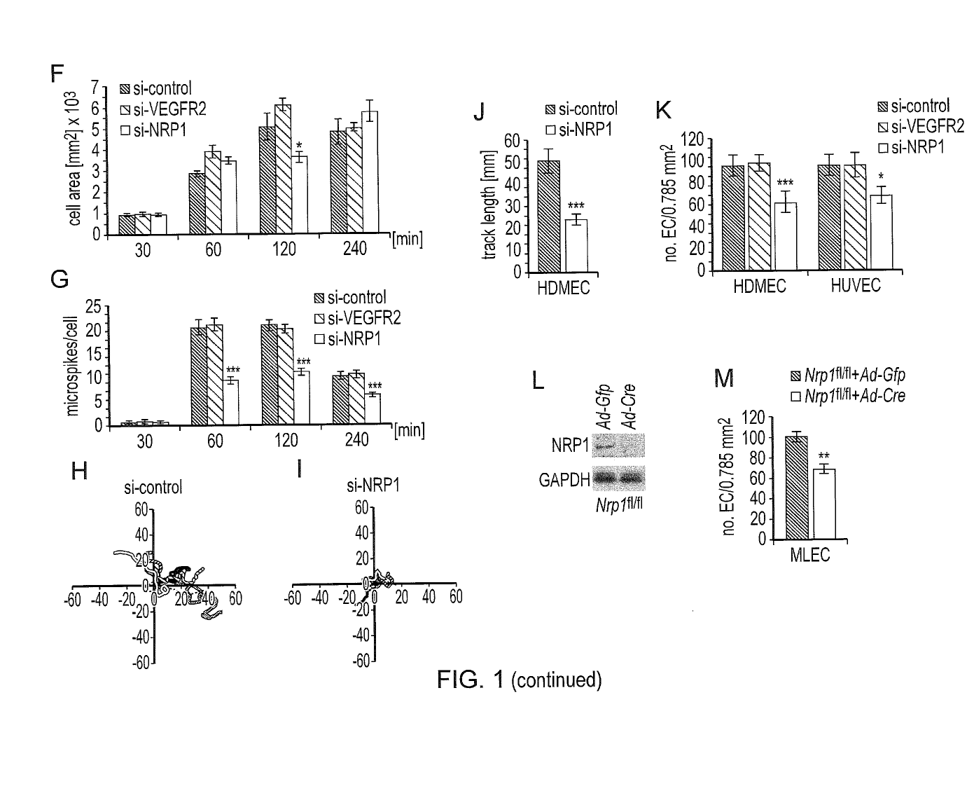

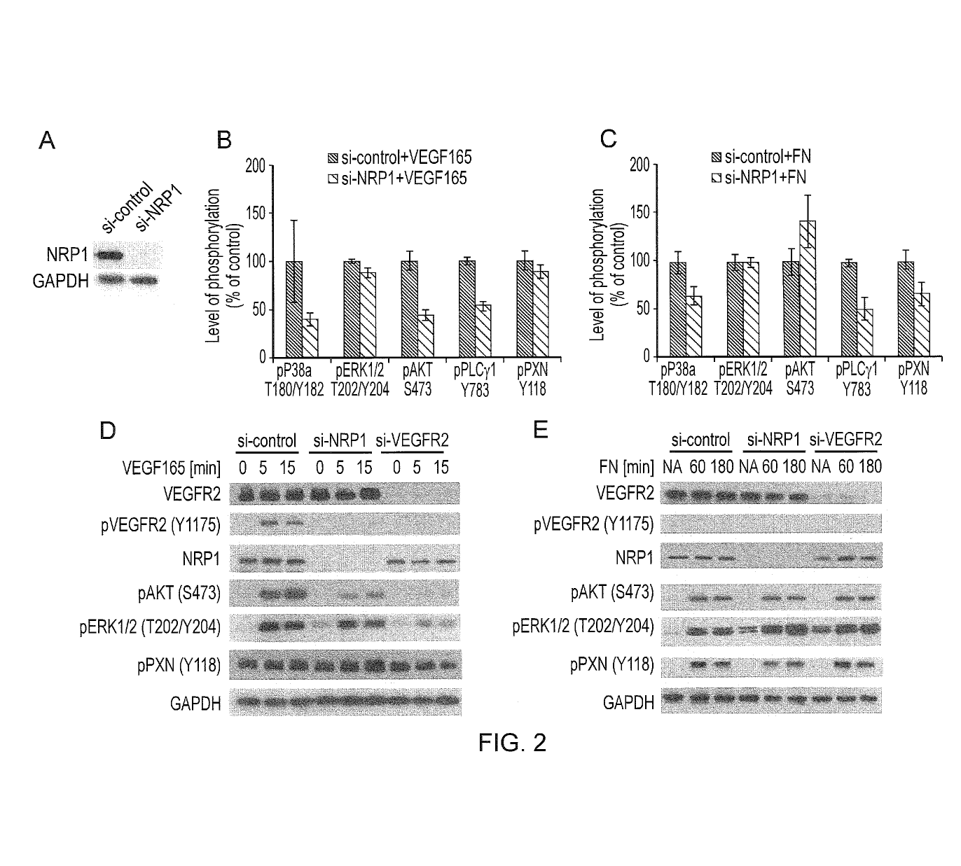

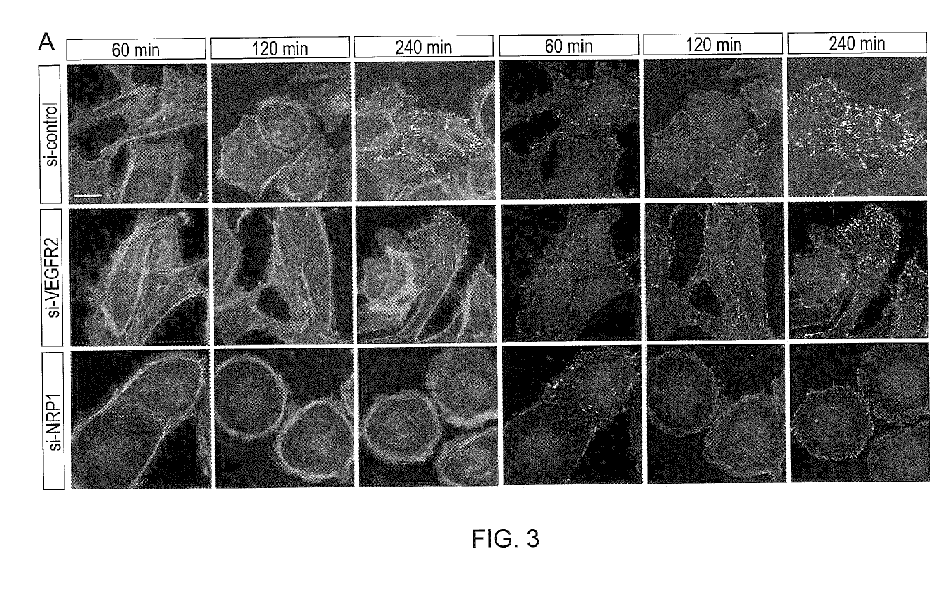

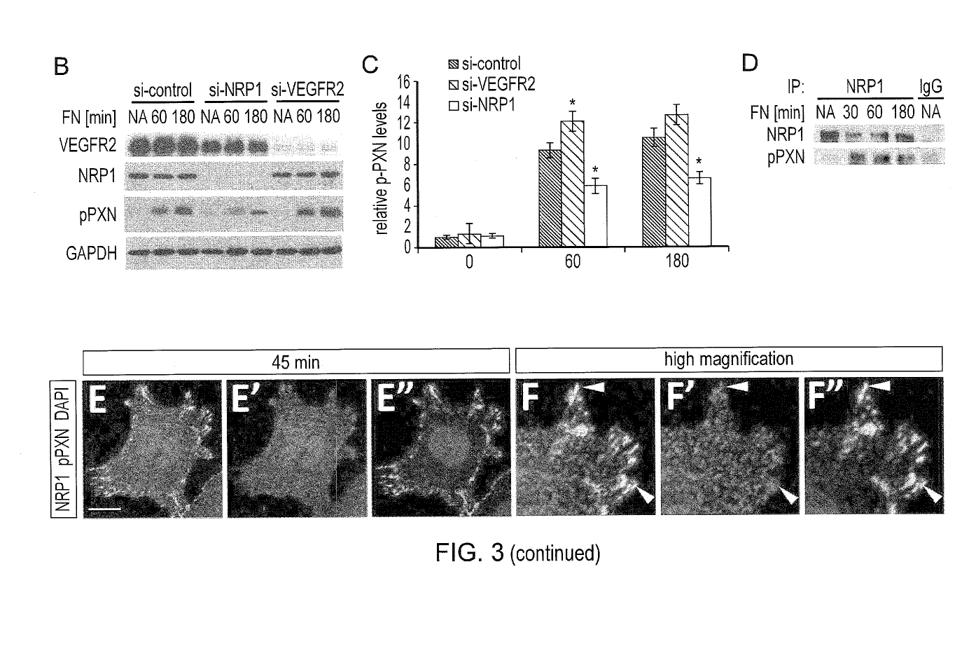

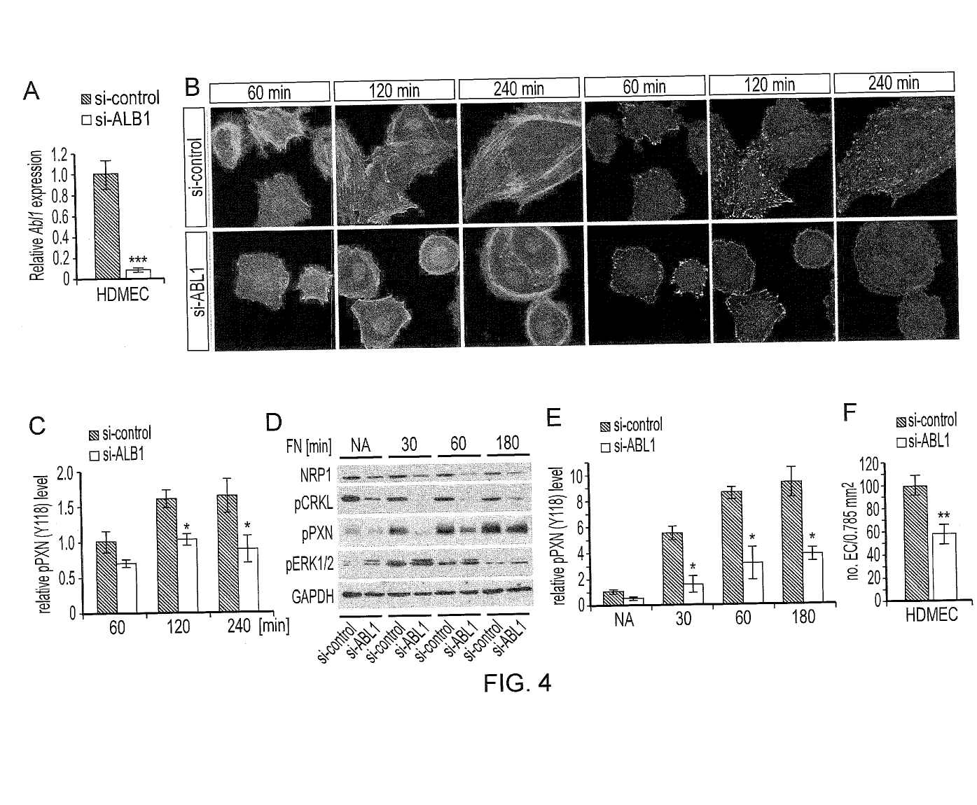

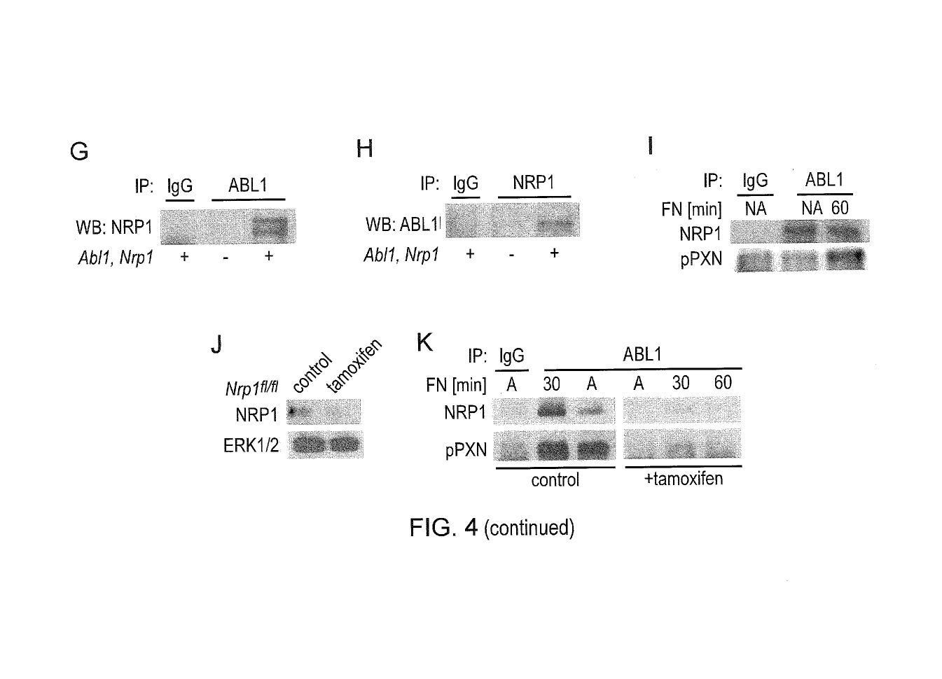

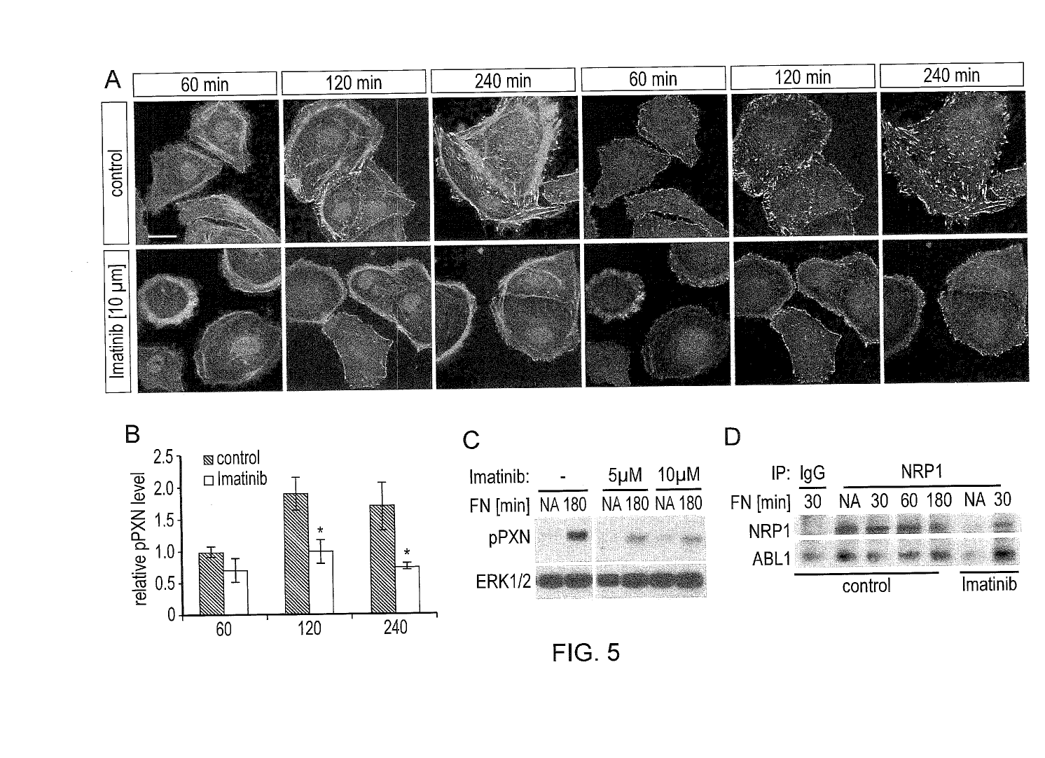

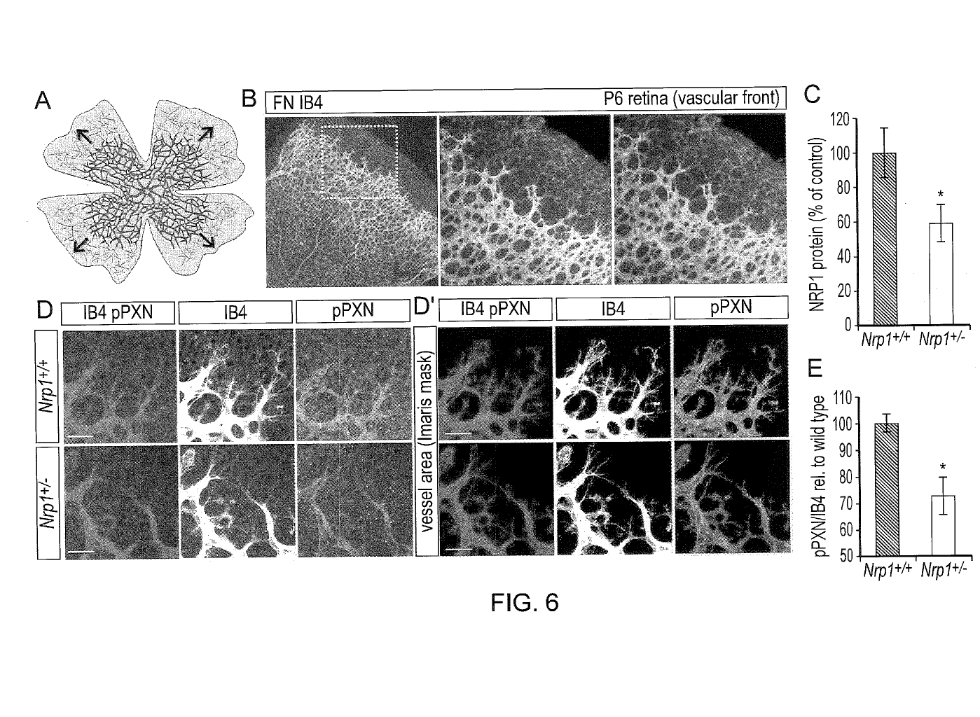

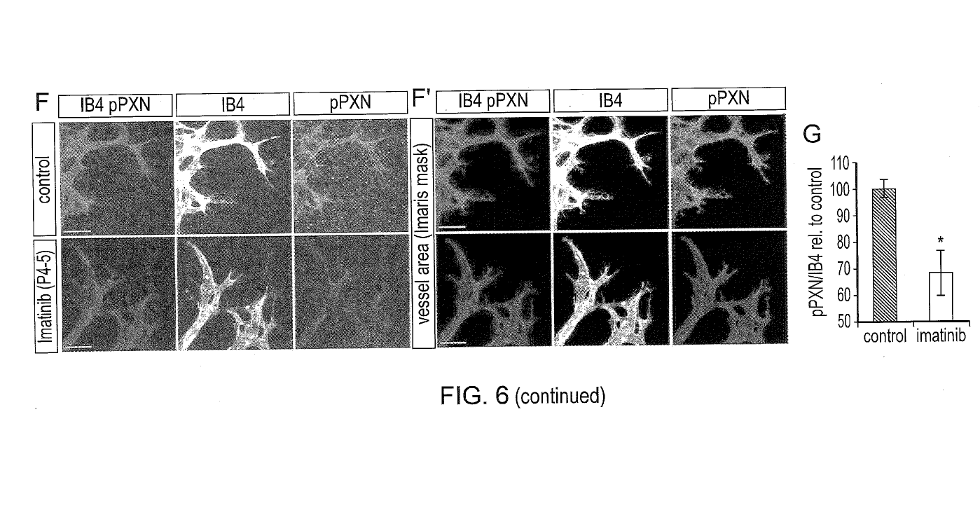

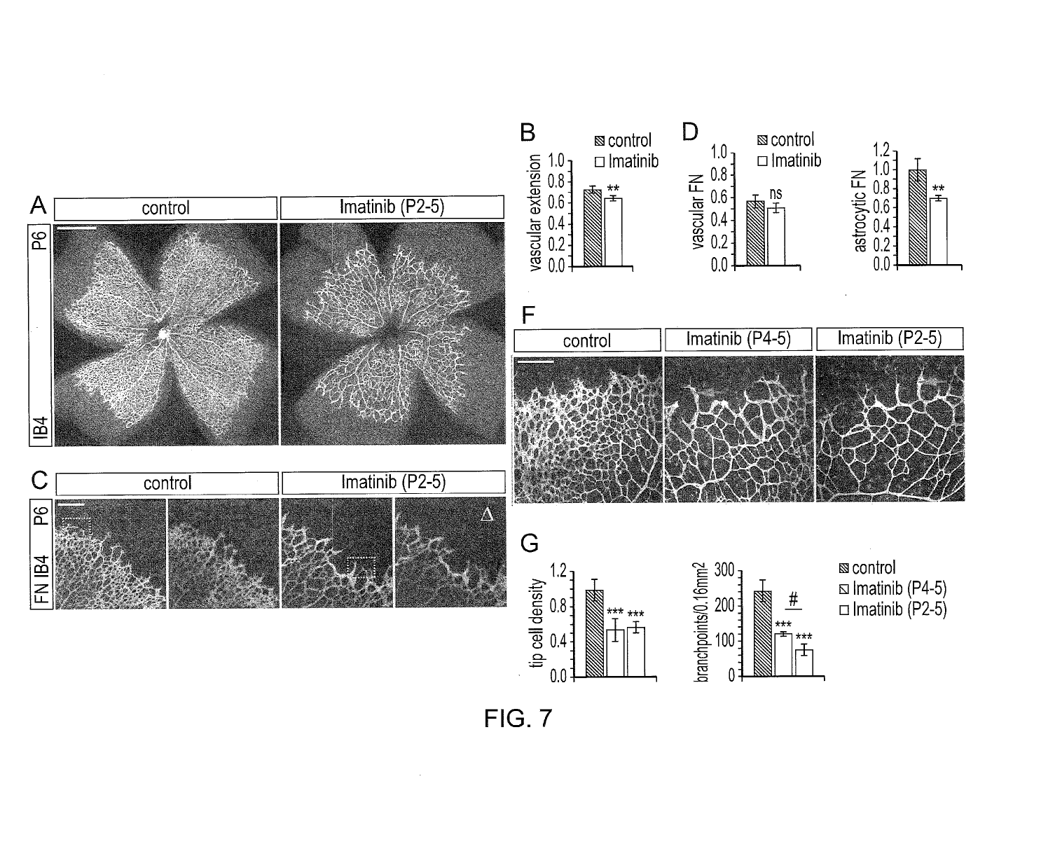

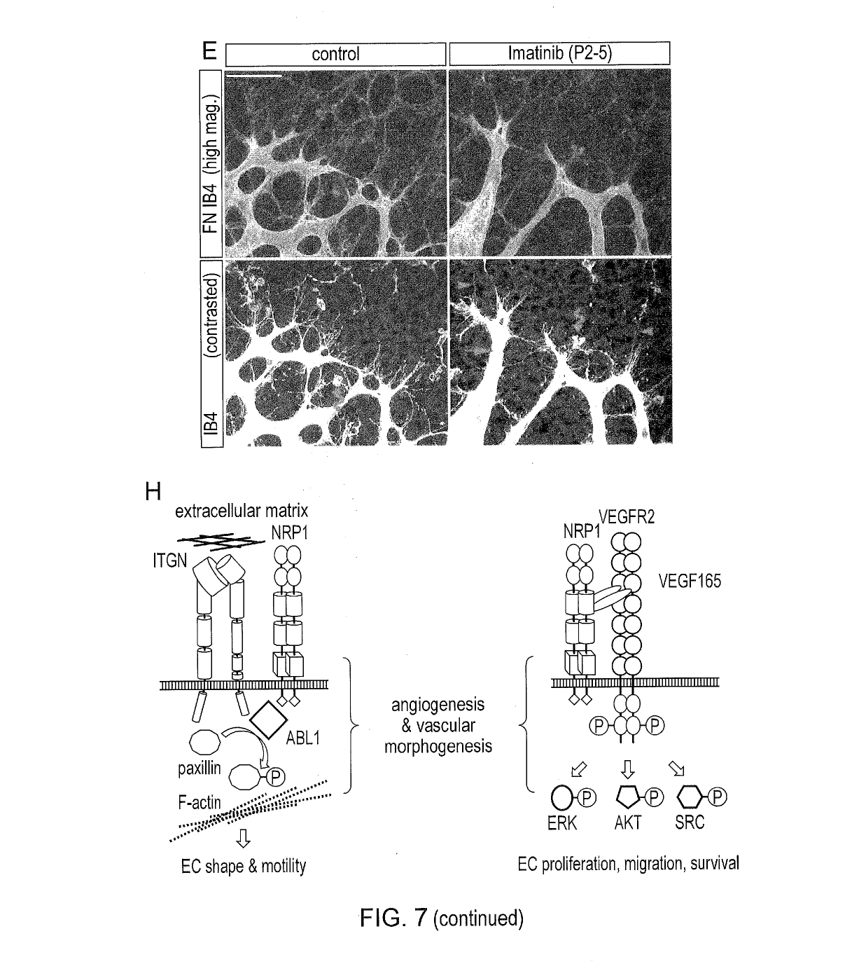

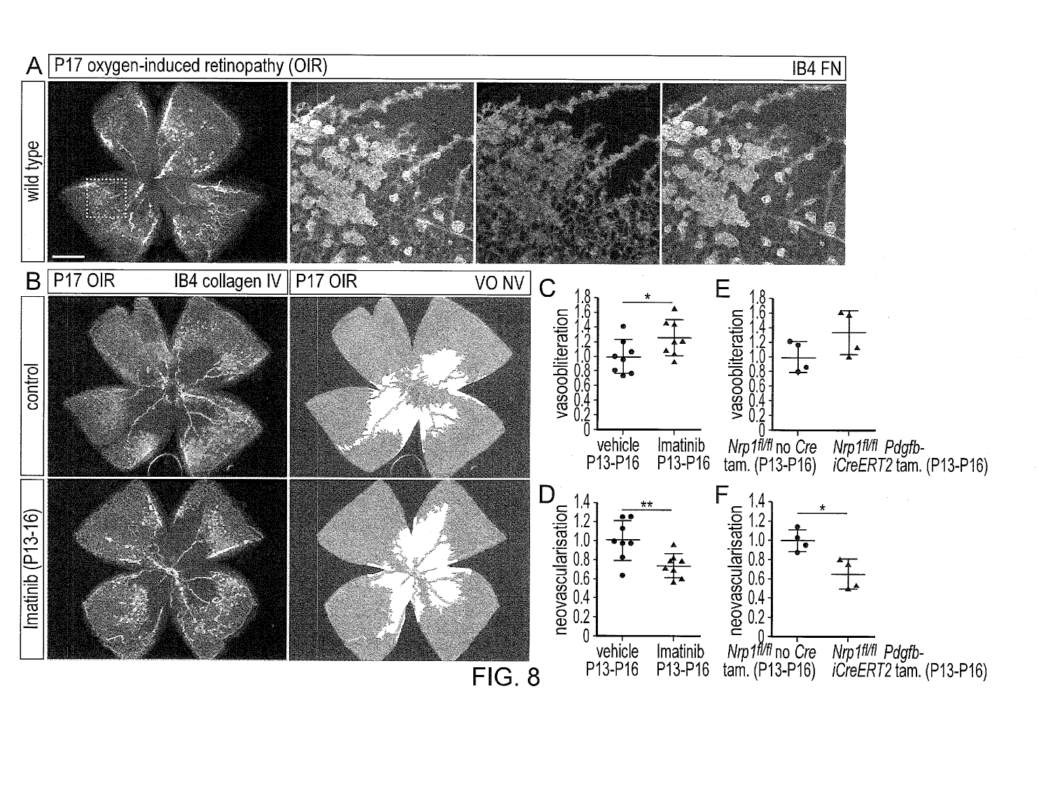

The requirement of NRP1 for endothelial cell (EC) adhesion, spreading and actin remodelling was examined. Human dermal microvascular EC (HDMEC) were used for these experiments because dermal vasculature naturally undergoes extensive angiogenesis during wound healing. HDMEC were transfected with a previously validated small interference (si) RNA that targets NRP1 or a control nonsense siRNA. Having confirmed the efficacy of this approach (FIG. 1A), HDMEC adhesion to tissue culture dishes coated with 10 μg/ml FN, a concentration that effectively promotes cell adhesion and migration, was tested, and it was determined that adhesion was not compromised by NRP1 deficiency (FIG. 1B,C). Adhesion of human umbilical cord EC (HUVEC) to plates coated with 10 μg/ml FN was also not impaired (data not shown). Conditions suitable to study FN-induced cell spreading, actin remodelling and cell migration of EC in the absence of prior defects in cell attachment were therefore identified.

Phalloidin staining of filamentous (F-) actin showed that NRP1-deficient HDMEC (FIG. 1D) adopted an abnormal round morphology with abundant cortical actin when plated on FN, whilst control cells appeared elongated and contained stress fibres typical of adherent cells (FIG. 1E). In contrast, HDMEC transfected with siRNA targeting VEGFR2 (FIG. 1D) spread well on FN and assembled many stress fibres (FIG. 1E). Quantitation confirmed that NRP1, but not VEGFR2 downregulation significantly impaired cell spreading on FN (FIG. 1F). High magnification images revealed phalloidin-positive, filopodia-like microspikes in HDMEC 60 and 120 min after plating on FN, which were significantly reduced after NRP1, but not VEGFR2 downregulation (FIG. 1E′, G). Moreover, addition of VEGF165, known to bind both VEGFR2 and NRP1, did not rescue the defects of FN-stimulated HDMEC lacking NRP1 (FIG. S1D, E).

NRP1 therefore functions independently of VEGF-A and VEGFR2 to regulate ECM-induced cell spreading and actin remodelling.

### Example 2

### NRP1 Promotes the Motility and Haptotactic Migration of FN-Stimulated EC

Tracking the behaviour of individual HDMEC after plating on FN demonstrated that control cells were significantly more motile than cells lacking NRP1 (FIG. 1H-J). Consistent with reduced motility, a transwell assay measuring ECM-induced haptotaxis demonstrated reduced migration of NRP1-deficient compared to control cells towards FN, but VEGFR2 knockdown did not affect migration (FIG. 1K). Similar results were obtained with HUVEC (FIG. 1K). To examine if NRP1 deficiency impaired EC migration in another species, mouse lung EC (MLEC) from Nrp1 conditional null (Nrp1^{fl/f1}) mice were used in the transwell assay after infection with adenovirus expressing GFP as a control or CRE recombinase to downregulate NRP1 (FIG. 1L). As observed for human EC, migration onto FN substrates was significantly reduced in MLEC lacking NRP1 compared to controls (FIG. 1M).

NRP1 deficiency therefore impairs ECM-induced EC motility and migration independently of VEGFR2.

### Example 3

### Identification of NRP1-Dependent ECM-Induced Signal Transduction Pathways

HDMEC were transfected with siNRP1 or control siRNA (FIG. 2A), and then serum-starved and stimulated with VEGF165 (FIG. 2B) or plated on FN (FIG. 2C). VEGF165-induced activation of the MAPK p38 in EC lacking NRP1 was reduced and additionally there was impaired FN-induced and NRP1-dependent P38 activation (FIG. 2B,C). The screen also showed that NRP1 downregulation in VEGF165-stimulated HDMEC reduced two other signal transduction pathways previously shown to operate downstream of growth factor signalling in EC, the MAPK kinase pathway involving ERK1/2 and the P13-kinase pathway activating AKT (FIG. 2B). Unexpectedly, however, NRP1 downregulation did not impair ERK1/2 and AKT phosphorylation in FN-simulated HDMEC (FIG. 2C).

NRP1 loss decreased both VEGF165- and FN-induced activation of PLCγ1 (FIG. 2B,C), known to promote the phosphorylation of the focal adhesion protein paxillin (PXN) downstream of FN-mediated integrin activation to increase cell spreading and migration. Consistent with reduced PLCγ1 activation and a requirement for NRP1 in integrin ligand-mediated EC spreading and motility, FN-induced PXN phosphorylation on tyrosine residue (Y) 118 was also reduced in FN-stimulated HDMEC lacking NRP1 (FIG. 2C). In contrast, VEGF165-induced PXN phosphorylation was unaffected (FIG. 2B).

The phosphoantibody screen therefore demonstrated essential and distinct roles for NRP1 in VEGF165- versus ECM-stimulated signal transduction.

### Example 4

### Differential Dependency of ECM Signalling Pathways on NRP1 and VEGFR2

Immunoblotting validated the results from the phosphoantibody screen and allowed direct the comparison of NRP1 and VEGFR2 dependence of specific signal transduction pathways (FIG. 2D,E). For these experiments, HDMEC were serum-starved and plated on FN or grown on tissue culture plastic, serum-starved and then stimulated with VEGF165. VEGF165 induced VEGFR2 phosphorylation was inhibited by NRP1 or VEGFR2 downregulation (FIG. 2D). In contrast, FN stimulation did not induce comparable VEGFR2 stimulation (FIG. 2E). Also, NRP1 or VEGFR2 knockdown decreased VEGF165-induced AKT phosphorylation; however, FN-induced AKT phosphorylation was unaffected by NRP1 or VEGFR2 deficiency (FIG. 2D,E). The AKT pathway therefore transduces growth factor, but not ECM signals in a VEGFR2- and NRP1-dependent fashion. VEGF165-, but not FN-induced ERK1/2 phosphorylation was also impaired after VEGFR2 knockdown (FIG. 2D,E). Finally, immunoblotting was used to examine how NRP1 and VEGFR2 knockdown affected PXN tyrosine phosphorylation. Whilst NRP1 loss reduced PXN phosphorylation in FN-stimulated HDMEC, VEGFR2 knockdown did not (FIG. 2E). Vice versa, PXN was constitutively phosphorylated in adherent HDMEC, with phosphorylation increasing slightly after VEGF165 stimulation in a VEGFR2-dependent manner (FIG. 2D), but NRP1 was not required for VEGF165-induced PXN phosphorylation (FIG. 2D).

These findings demonstrate that ECM- and VEGF165-induced signal transduction pathways are differentially affected by NRP1 loss, and that NRP1-dependent PXN phosphorylation occurs independently of VEGFR2.

### Example 5

### NRP1, not VEGFR2, is required for ECM-induced PXN phosphorylation and focal adhesion localisation

NRP1-dependent pPXN localisation in FN-stimulated HDMEC was examined by immunostaining. In control cells, pPXN increased over time and was present in focal adhesions at the end of F-actin stress fibres, correlating with an elongated cell shape (FIG. 3A). FN-stimulated HDMEC therefore displayed the hallmarks of polarised cells. Whilst VEGFR2 downregulation did not impair these responses, HDMEC lacking NRP1 displayed their characteristic rounded morphology with abundant cortical actin; correlating with the lack of stress fibres, pPXN levels were significantly decreased, with remaining pPXN being localised mainly to the cell periphery (FIG. 3A,B). Immunoblotting established that siRNA targeting had been effective and further confirmed reduced pPXN in NRP1-deficient cells; in contrast, pPXN was slightly, but significantly increased in VEGFR2-deficient cells (FIG. 3B,C). These findings demonstrate that NRP1 promotes PXN phosphorylation in a VEGFR2-independent fashion. NRP1 immunoprecipitation followed by pPXN immunoblotting demonstrated a FN-dependent interaction in HDMEC (FIG. 3D). Moreover, immunofluorescence showed partial co-localisation in peripheral cell areas resembling focal adhesions (FIG. 3E,F).

### Example 6

### ABL1 is Essential for ECM-Induced PXN Phosphorylation

To investigate ABL1 function in FN-stimulated EC, two independent, complementary methods; siRNA-mediated knockdown of ABL1 (FIG. 4) and pharmacological inhibition of ABL1 kinase activity were utilised (FIG. 5).

ABL1 knockdown was achieved by transfecting HDMEC with control siRNA or siRNA targeting ABL1 (siABL1) and confirmed by qPCR (FIG. 4A). Targeting ABL1 caused a phenotype similar to NRP1 knockdown. Thus, immunostaining revealed significantly reduced pPXN levels, with residual pPXN accumulating in the cell periphery (FIG. 4B,C). Moreover, there was a conspicuous absence of pPXN-positive focal adhesion contacts in areas where stress fibres terminate in control cells, but abundant cortical actin, correlating with impaired cell spreading and a round, rather than elongated cell shape, as observed after NRP1 knockdown (FIG. 4B). Immunoblotting for the phosphorylated form of the ABL1 target CRKL confirmed ABL1 downregulation and significantly reduced FN-stimulated pPXN in ABL1 knockdown compared to control cells (FIG. 4D,E). This analysis also showed increased ERK1/2 phosphorylation after ABL1 knockdown (FIG. 4D), similar to NRP1 knockdown (FIG. 2D). A transwell assay showed a 40% reduction in haptotactic migration after ABL1 knockdown (FIG. 4F). NRP1 and ABL1 therefore similarly regulate PXN phosphorylation, actin remodelling and cell migration in FN-stimulated EC.

### Example 7

### ABL1 Forms a Complex With NRP1 in EC

HEK293 cells were co-transfected with expression constructs for NRP1 and ABL1. ABL1 immunoprecipitation followed by NRP1 immunoblotting and vice versa showed that both proteins formed a complex (FIG. 4G-H). Immunoprecipitating ABL1 from HDMEC followed by NRP1 immunoblotting confirmed the existence of an endogenous complex and further showed that this complex had recruited pPXN 60 min after FN stimulation (FIG. 4I). Two conditional Nrp1-null alleles were inactivated in newborn mice with a tamoxifen-inducible endothelial Cre transgene, and then EC were isolated (FIG. 4J). As observed for HDMEC, ABL1 immunoprecipitated NRP1 in control MLEC adhering to FN, and complex formation was increased after detaching the cells and plating them for 30 min on FN (FIG. 4K). Moreover, pPXN was present in the ABL1-NRP1 complex of MLEC from both conditions (FIG. 4K), as seen in HDMEC (FIG. 4I). In contrast, NRP1 was not immunoprecipitated effectively in MLEC from tamoxifen-treated mice, and complex formation of ABL1 with pPXN was reduced (FIG. 4K). These data demonstrate that ABL1 and NRP1 form a complex and that ABL1 recruits pPXN in a NRP1-dependent fashion.

### Example 8

### ABL1 Kinase Inhibition Reduces ECM-Induced PXN Phosphorylation

HDMEC were treated with Imatinib, a small molecule inhibitor that effectively targets ABL1, but not VEGFR2, and has been approved for therapy in cancers with upregulated ABL1 kinase activity. As observed in siABL1-transfected cells, lmatinib-treated HDMEC formed few stress fibres, but abundant cortical actin and adopted a round shape with reduced cell spreading; moreover, they had low pPXN phosphorylation, with residual pPXN in the cell periphery rather than in areas where stress fibres normally terminate in focal adhesions (FIG. 5A). The quantitation of pixel intensities in immunostains (FIG. 5A,B) and immunoblots (FIG. 5C) confirmed significantly reduced PXN phosphorylation in lmatinib-treated compared to control cells. NRP1 immunoprecipitation of Imatinib-treated HDMEC followed by ABL1 immunoblotting showed that both proteins formed a complex prior to FN stimulation, independently of ABL1 kinase activity (FIG. 5D).

### Example 9

NRP1 regulates ABL1-mediated PXN phosphorylation in retinal angiogenesis

To examine if NRP1 and ABL1 also promote PXN phosphorylation in an ECM-dependent angiogenesis model in vivo, the perinatal mouse retina was studied. In this organ, endothelial sprouts headed by filopodia-studded tip cells migrate towards astrocyte-localised VEGF-A in the retinal periphery, with filopodia being guided by astrocyte-derived FN (FIG. 6A). Immunostaining confirmed FN deposition around IB4-stained vessels and ahead of the vascular front in a fine meshwork characteristic of astrocytes (FIG. 6B). Nrp1^{-/-} mice could not be used to investigate how NRP1 deficiency affected pPXN levels during retinal angiogenesis, as they die before birth; instead, Nrp1^{+/+} and Nrp1^{+/-} littermates were used, because the latter are viable, but nevertheless have mild angiogenesis defects. Immunoblotting confirmed significantly decreased NRP1 levels in P6 Nrp1^{+/-} mutants (FIG. 6C). Single optical slices, acquired by confocal microscopy after immunolabelling, revealed pPXN staining in EC at the IB4-positive vascular front, including tip cells and their filopodia, and also some pPXN ahead of the vascular front (FIG. 6D). As observed for siNRP1-targeted HDMEC, pPXN staining appeared reduced in Nrp1^{+/-} littermates, both in vascular and avascular areas (FIG. 6D). To quantify pPXN specifically in EC, IB4-positive areas were isolated by applying a virtual mask over IB4-negative areas (FIG. 6D′). This method also excluded IB4-positive macrophages, which interact with endothelial tip cells to promote vascular sprout fusion. Quantification of pPXN relative to IB4 pixel intensity in masked 3D-projections of z-stacks showed significantly reduced endothelial pPXN staining at the vascular front of heterozygotes (FIG. 6E). To examine if ABL1 was also required for PXN phosphorylation in retinal EC, neonates were treated with Imatinib or vehicle (FIG. 6F). Single confocal slices showed that pPXN staining was reduced in P6 retinas after 2 days of treatment, as observed for Nrp1^{+/-} mutants (FIG. 6D). To quantify pPXN specifically in EC, IB4-positive areas were again isolated with a virtual mask (FIG. 6F′) and it was found that endothelial pPXN pixel intensity was significantly reduced after lmatinib treatment (FIG. 6G).

ABL1 therefore promotes PXN phosphorylation in angiogenic EC in vivo, similar to NRP1 and as observed for FN-stimulated EC in vitro.

### Example 10

### ABL1 Activation is Essential for Physiological Angiogenesis in the Retina

IB4 labelling showed reduced vascular extension and network density in retinal flatmounts of P6 Imatinib—compared to vehicle-treated mice (FIG. 7A). Quantitation confirmed a small, but significant reduction in vascular extension across the retina (FIG. 7B). FN immunostaining showed reduced astrocyte FN deposition ahead of the vascular front (FIG. 7C,D), likely due to lmatinib-targeting of PDGFR signalling in astrocytes. In contrast, abundant FN deposition was observed around retinal vessels in both control and Imatinib-treated mice, with no difference in FN pixel intensity in IB4-positive areas (FIG. 7C,D). Vascular FN assembly was therefore not compromised by ABL1 targeting.

High magnification images of the retinal vascular front in control and Imatinib-treated mice showed that vessels had sprouted and formed numerous tip cell filopodia (FIG. 7E), consistent with the concept that VEGF-A gradients rather than matrix pathways drive these processes. However, sprouts in Imatinib-treated mice appeared longer and wider, with fewer lateral protrusions connecting to neighbouring sprouts (red arrows, FIG. 7E,F), and filopodia often appeared thin, wavy and misoriented (red arrowheads, FIG. 7E). This phenotype agrees with a role for the NRP1-ABL1 complex in endothelial actin remodelling (FIG. 1). Moreover, the filopodia defects resemble those previously seen at the retinal vascular front of mice with a genetic lack of astrocyte FN. Yet, loss of astrocyte FN in itself was previously shown not to impair vessel sprouting and branching, suggesting that the more severe vascular defects of lmatinib-treated retinas are due to an endothelial cell-autonomous role for ABL1. To further define the vascular defects caused by ABL1 inhibition, vessel sprouting and branching were compared in mice treated with Imatinib for different times (FIG. 7F,G). Quantitative analysis revealed significantly fewer tip cells, defined by the presence of filopodial bursts at the vascular front (FIG. 7G). Consistent with reduced tip cell density and impaired lateral sprout extension, a significant and lmatinib dose-dependent reduction in branchpoints behind the vascular front was observed, indicative of reduced sprout fusion (FIG. 7G).

### Example 11

### Imatinib Treatment or Loss of Endothelial NRP1 Similarly Impair Pathological Angiogenesis in the Retina

To assess the relevance of the FN-induced NRP1-ABL1 pathway for pathological vessel growth, a mouse model of oxygen-induced retinopathy (OIR) was used (Connor et al., 2009). In this model, the sequential exposure of mouse pups to hyperoxia followed by normoxia leads to the formation of retinal neovascular lesions that resemble those observed in PDR patients and in babies with ROP, which arises after moving them out off incubators with high oxygen tension. Specifically, the exposure of neonatal mice to hyperoxia causes vasoobliteration (VO) of central retinal capillaries, which causes central retinal hypoxia on return to room air. The ensuing upregulation of VEGF and other proangiogenic factors then activates angiogenesis, but this process fails to effectively revascularise the retina and instead leads to abundant tuft-like vascular malformations that protrude into the vitreous. Immunostaining showed that these neovascular tufts were FN-positive (FIG. 8A). Imatinib treatment of mouse pups from their return to normoxia until P17 slightly reduced revascularisation of areas affected by VO (FIG. 8B,C), but effectively inhibited the formation of neovascular tufts (FIG. 8B-D). When endothelial NRP1 expression was targeted by Cre-LoxP recombination with a tamoxifen regiment analogous to that used for Imatinib treatment, a similar mild reduction in revascularisation of avascular areas (FIG. 8E) and pronounced reduction in neovascular tufts (FIG. 8F) was observed

Materials and Methods

Cell Culture, Transfection and Adenovirus Infection

Human microvascular dermal EC (HDMEC; Promocell, UK) and human umbilical vein EC (HUVEC; Promocell) were cultured in standard conditions and transfected with Lipofectamine RNAIMAX (Life Technologies). Primary mouse lung EC (MLEC) were isolated from mice between one and two months of age by magnetic-activated cell sorting (MACS) with PECAM antibodies (BD Biosciences, UK). In some experiments, cells were incubated with 10 μM Imatinib (Cambridge Bioscience, UK), a concentration known to effectively target ABL1 kinase (Chislock et al., 2013). In some experiments, cells were stimulated with VEGF165 50 ng/ml for the indicated times.

Adhesion, Motility and Cell Migration Assays

For adhesion assays, transfected HDMEC or HUVEC were seeded on 96-well tissue culture plates (Nunc, Thermoscientific, UK) coated overnight with 10 μg/ml FN in PBS or, as an internal control, PBS containing 1% BSA; for migration assays, transfected HDMEC or MLEC treated with adenovirus were serum-starved overnight and then plated onto FN-coated transwell. To measure random motility, transfected HDMEC were imaged with a 10× phase contrast objective at 2 min intervals for 200 minutes using an inverted phase contrast microscope (Axiovert-200M, Carl Zeiss, UK).

Phospho-Kinase-Antibody Array

Transfected HDMEC were serum-starved and stimulated with VEGF or seeded on FN before being lysed; the phospho-kinase-antibody array was performed according to the manufacturer's instructions (R&D Systems, UK).

Gene Expression

Gene expression was analysed with the quantitative 7500 Real-Time PCR System (Applied Biosystems).

Immunoblotting and Immunoprecipitation

Protein extracts were analysed by SDS-PAGE and transferred to nitrocellulose membrane (Whatman, USA) for immunoblotting with the following primary antibodies: rabbit anti-GAPDH (Abcam, UK), goat anti-NRP1 C-19 (Santa Cruz Biotechnology, USA) or AF566 (R&D Systems); mouse anti-ABL1 (BD Bioscience, UK); rabbit anti pKDR (Y1175) or KDR, rabbit anti-pPXN (Y118), rabbit anti-pAKT (S473) or AKT, rabbit anti-pERK1/2 or ERK1/2 or pCRKL (Y207) (Cell Signaling Technology, USA); NRP1, ABL1 and PXN were immunoprecipitated from HDMEC or MLEC seeded on FN in the presence or absence of lmatinib.

Animals

All animal procedures were performed in accordance with institutional and UK Home Office guidelines. We used C57/616 wild type mice (Charles River Laboratories), littermate wild type and heterozygous Nrp/-null mutants and mice carrying two floxed conditional null Nrp1 alleles (Nrp1^{fl/fl}) combined with Pdgfb-iCre-ERT2-Egfp with codon-improved Cre on a C57/616 background. For tamoxifen-induction of CRE-mediated recombination, 0.2 mg of tamoxifen (SIGMA) dissolved in peanut oil to 2 mg/ml was administered via intraperitoneal injection into pups at the indicated times. In some experiments, mice were injected intraperitoneally with 100 mg/kg/day Imatinib in 0.9% saline solution for the indicated times. OIR was induced as previously described. Briefly, nursing dams and their pups were maintained at 75±3% O₂ in an oxygen supply chamber from P7 to P12; dams were rested in room air for 1 h each day. Dams and pups were returned to room air on P12 and the pups culled on P17 to isolate retinas for immunolabelling; the size of the avascular and neovascular areas was determined with Photoshop CS4 (Adobe Inc.) in images acquired with a SZX16 stereofluorescent microscope (Olympus) equipped with a digital camera (Hamamatsu Photonics Ltd).

Immunofluorescence:

Immunofluorescence staining of HDMEC and p6 mouse retinas was analysed with a L5M710 laser scanning confocal microscope.

Statistical Analyses:

Statistical analyses were performed with GraphPad (Prism) or Office Excel (Microsoft); data obtained from cell lines were analysed with a two-tailed, paired t-test, whilst data from retina samples were analysed using a two-tailed, unpaired t-test or ANOVA.

1. An Abelson murine leukaemia viral oncogene homolog 1 (ABL1) inhibitor for use in the treatment and/or prevention of ocular neovascularisation associated with a non-cancerous condition.

2. An ABL1 inhibitor according to claim 1 for use in the treatment and/or prevention of ocular neovascularisation associated with one or more of the following conditions:
age-related macular degeneration, diabetic retinopathy, macular telangiectasia, choroidal neovascularisation or diabetic macular oedema, retinal vein occlusion, retinopathy of prematurity, ocular histoplasmosis and pathologic myopia macular oedema.

3. An ABL1 inhibitor according to claim 2 for use in the treatment and/or prevention of age-related macular degeneration, diabetic retinopathy or diabetic macular oedema.

4. An ABL1 inhibitor for use according to any preceding claim wherein the inhibitor inhibits angiogenesis.

5. An ABL1 inhibitor for use according to any preceding claim wherein the inhibitor inhibits endothelial cell actin remodelling and/or migration.

6. An ABL1 inhibitor for use according to any preceding claim wherein the inhibitor inhibits neuropilin 1 (NRP1)-dependent signalling.

7. An ABL1 inhibitor for use according to any preceding claim wherein the inhibitor inhibits paxillin (PXN) phosphorylation.

8. An ABL1 inhibitor for use according to any preceding claim wherein the inhibitor is selected from the following group: Imatinib, nilotinib, dasatinib, bosutinib, ponatinib, bafetinib and GNF-5.

9. An ABL1 inhibitor for use according to claim 8, wherein the inhibitor is Imatinib.

10. An ABL1 inhibitor for use according to claim 8, wherein the inhibitor is GNF-5.

11. An ABL1 inhibitor for use according to any preceding claim wherein the inhibitor is used in combination with a vascular endothelial growth factor (VEGF) or VEGF receptor inhibitor.

12. An ABL1 inhibitor for use according to claim 11 wherein the VEGF inhibitor inhibits the binding of VEGF to VEGF receptor or signalling of VEGF through VEGF receptor.

13. An ABL1 inhibitor for use according to claim 11 wherein the VEGF inhibitor is Avastin, Lucentis, Eylea or Macugen.

14. An ABL1 inhibitor for use according to any of claims 11 to 13 wherein the inhibitor and the VEGF or VEGFR inhibitor are administered simultaneously, separately or sequentially.

15. A pharmaceutical composition comprising an ABL1 inhibitor for use in the treatment and/or prevention of ocular neovascularisation associated with a non-cancerous condition.

16. A pharmaceutical composition according to claim 15 wherein the composition also comprises a pharmaceutically acceptable excipient and/or carrier.

17. A pharmaceutical composition according to claim 15 or 16 wherein the composition comprises a VEGF or VEGF receptor inhibitor.

18. A kit comprising an ABL1 inhibitor and a VEGF or VEGF receptor inhibitor for use in the treatment and/or prevention of ocular neovascularisation associated with a non-cancerous condition.

19. The kit according to claim 18 wherein the VEGF or VEGF receptor inhibitor and the ABL1 inhibitor are for separate, subsequent or simultaneous administration.

20. A method for the treatment and/or prevention of ocular neovascularisation associated with a non-cancerous condition in a subject, which comprises the step of administering an ABL1 inhibitor to the subject.

21. The use of an ABL1 inhibitor in the manufacture of a medicament for treating and/or preventing ocular neovascularisation associated with a non-cancerous condition in a subject.
